(19) United States
(12) Patent Application Publication
(10) Pub. No.: US 2005/0131301 A1
(43) Pub. Date: Jun. 16, 2005

(54) Ultrasound probe receptacle

(76) Inventors: Michael Peszynski, Newburyport, MA (US); Douglas Thoreson, Woodinville, WA (US); Mark Dieselman, Amesbury, MA (US)

Correspondence Address: PHILIPS INTELLECTUAL PROPERTY & STANDARDS, P.O. BOX 3001, BRIARCLIFF MANOR, NY, 10510, US

(21) Appl. No.: 11/007,379

(22) Filed: Dec. 8, 2004

Related U.S. Application Data
(60) Provisional application No. 60/529,122, filed on Dec. 12, 2003.

(51) Int. Cl. A61B 8/14

(52) U.S. Cl. 600/459

(57) ABSTRACT

A receptacle stores an ultrasonic transducer used for ultrasound imaging in critical or semi-critical environments. The receptacle is also used for transporting the ultrasonic transducer between an examining area and a designated cleaning/disinfecting area, and, in addition, provides appropriate sanitary safeguards for the ultrasonic transducer. The ultrasound system operator can secure the receptacle to the ultrasound system in order to store or hold the ultrasonic transducer. After the ultrasonic transducer has been used, it is placed in the receptacle, which is detached and brought to the cleaning/disinfecting area. The receptacle may have a cover and an inner lining for protecting the ultrasonic transducer, as well as a status indicator so that the operator can quickly determine the present condition of the transducer.

### CROSS REFERENCE TO RELATED CASES

Applicants claim the benefit of Provisional Application Ser. No. 60/529,122, filed DEC. 12, 2003.

### BACKGROUND OF THE INVENTION

1. Field of the Invention

This invention relates generally to storing transducers used in ultrasonic imaging systems and, in particular, to the storage and maintenance of endocavity transducers, such as transesophageal echo (TEE) transducers.

2. Description of the Related Art

Ultrasonic imaging systems are used to observe the internal organs of a patient. The ultrasonic range is described essentially by its lower limit: 20 kHz, roughly the highest frequency a human can hear, although medical ultrasound imaging systems tend to use frequencies in the 2 to 15 MHz range. The systems emit ultrasonic pulses which, if not absorbed, echo (i.e., reflect), refract, or are scattered by structures in the body. Most of the received signal is from scattering, which is caused by many small inhomogeneities (much smaller than a wavelength) making a small part of the wave energy disperse in all directions. The signals are received and are translated into images by the ultrasound system.

A typical ultrasound system 100 is shown in FIG. 1, with each component labeled. The system 100 shown in FIG. 1 is a SONOS 7500/5500 ultrasound system from Philips Electronics (Philips Ultrasound, Andover, Mass.). As can be seen in the drawings, ultrasound system 100 is mounted on four wheels 110 so that it can be relatively easily moved by a single person. These wheels can be locked in place by wheel lock 112 when sonography is being performed on a patient. The operator of the system uses keyboard 120 and touch panels 122 to control the operation of ultrasound system 100, and can also use optical disk drive 132, floppy disk drive 134, or videocassette recorder (VCR) 136, to store data and/or images from system 100. A monitor 140 displays the ultrasound images.

The component which is placed on (or in) the patient in order to transmit and receive the ultrasonic waves which image the region of interest in the patient is called a “transducer”. In FIG. 1, several ultrasonic transducers 150 are shown being held by transducer holders 153 on the right-hand side of system 100, next to monitor 140. Although shown in the particular configuration of FIG. 1, it should be noted that transducer holders may take a variety of forms and may be located at a variety of places within and/or around the ultrasound system. In order to communicate data back to ultrasound system 100, the various ultrasound transducers 150 are connected by a cable to transducer connections 155 on ultrasound system 100.

Ultrasonic transducers take a variety of shapes and sizes, and each one is designed for one or more particular imaging applications. In general, transducers fit in one of two categories: those used externally (placed on the skin of the patient) and those used internally (placed inside the patient). External ultrasonic transducers, such as those used to perform imaging of a fetus during pregnancy, are familiar to most of the public. Internal ultrasonic transducers are less well-known, and include endocavity transducers (such as endorectal and endovaginal transducers) and intraoperative transducers (which are used during surgery to visualize internal structures).

One type of endocavity transducer is a transesophageal echo (TEE) transducer, such as the TEE transducer 200 shown in FIG. 2. The ultrasonic transducer probe 210 located at the distal end of TEE transducer 200 contains the transducer elements which transmit and receive the ultrasonic waves. Probe 210 is inserted into the patient's esophagus in order to scan internal organs, such as the heart, from its location inside the esophagus (thus having a vantage point which is not blocked by bone or other tissue). Deflection area 220 which connects probe 210 to flexible shaft 230 is capable of movement (i.e., “deflection”) relative to flexible shaft 230 so that TEE probe 210 can be aimed at the appropriate area. At the other end of flexible shaft 230 is probe handle 240 which contains the deflection controls for deflection area 220. At its other end, probe handle 240 is connected to system connector cable 250 which is used to connect transducer 200 to system 100 by means of connector plug 260. Connector plug 260 plugs into one of the transducer connections 155 in FIG. 1.

A TEE transducer 200, with its probe 210 inserted inside a patient, can be seen in FIG. 3, which is based on FIG. 8 of U.S. Pat. No. 6,572,547 to Miller et al., assigned to the same assignee as the present invention and hereby incorporated by reference. In FIG. 3, probe 210 is shown inside the esophagus 380 of a patient. Flexible shaft 230 leads from the deflection area 220 up through the esophagus 380 and the mouth 330 of the patient to probe handle 240 located outside of the patent's mouth 330. The operator uses probe handle 240 to control the relative orientation of probe 210 inside the esophagus 380. The flexible shaft 230 is inserted through introducer 335 which extends from the patient's lips back to the patient's uvula 333.

In FIG. 3, introducer 335 protects the flexible shaft 230 from the patent and protects the patient from flexible shaft 230. Although an introducer is shown in FIG. 3 (and described in the '547 patent), most ultrasound TEE examinations are performed without an introducer (except in special cases, such as infants). In most ultrasound TEE examinations, a bite guard is used to protect the transducer probe. It is true that operators may perform the TEE examination without a bite guard, but this is not recommended, as damage to the TEE transducer may result. Although seen in one location inside esophagus 380, probe 210 can be inserted further into the patient, i.e., into the fundus of the stomach (see, e.g., FIG. 8C of the '547 patent). Ultrasonic transducer array 342 on probe 210 transmits and receives the ultrasonic waves as seen by the dotted lines in FIG. 3.

Although TEE transducer 200 in FIGS. 2 and 3 resembles the Omniplane series of TEE transducers from Philips Electronics (Philips Ultrasound, Andover, Mass.), the TEE transducer 200 in FIGS. 2 and 3 is intended only as an example of any endocavity transducers which may be used with any ultrasound system. Similarly, the ultrasound system in FIG. 1 is intended to represent any ultrasound system which uses endocavity transducers.

Because, as graphically depicted by TEE transducer 200 in FIG. 3, endocavity transducers are inserted deeply into the bodies of patients, a protective disposable sheath is typically used to cover the endocavity transducer during an examination. Furthermore, endocavity transducers must be carefully maintained, cleaned, and disinfected between uses.

Exemplary transducer cleaning and maintenance operations are described in SONOS 7500/5500 TRANSDUCER REFERENCE GUIDE, Edition 7, published in December 2002 by Philips Electronics (Part No. M2424-99300-07; hereinafter referred to as “GUIDE”), hereby incorporated by reference. For example, as described therein, an endocavity transducer must be thoroughly washed, preferably with an enzymatic cleaner, and disinfected, preferably with a glutaraldehyde-based disinfectant, after each examination. After disinfecting the transducer for a period of time, it is thoroughly rinsed with sterile water and checked for any residual organic material. If any is found, the process is repeated (for full description, see, e.g., pages 2-14-2-15, GUIDE).

Although endocavity transducers could be stored like the external transducers 150 in FIG. 1, i.e., in holders 153 on the side of the ultrasound system 100, this is not recommended for a number of reasons, some of which are obvious. First, storing endocavity transducers in such external holders is ill-advised because the surfaces of the endocavity transducer would be exposed to the environment, which may include direct sunlight and/or temperature extremes, as well as contaminants from the open air and accidental contact with other objects. Second, endocavity transducers must be stored separately from the other transducers in order to avoid any potential damage (see, e.g., page 2-4, GUIDE). Because of these dangers (and others), many operators will store endocavity transducers in their original case or in a drawer. However, this is also inappropriate, as there may be contamination if the transducer is stored in a slightly damp condition, and neither a typical drawer nor the original packaging for the transducer is designed for storing a cleaned and disinfected endocavity transducer until its next use.

As an example of the storage problems for endocavity transducers, consider a TEE transducer, such as the one described in reference to FIGS. 2 and 3. A new TEE transducer typically arrives in a carrying case, such as the one shown in FIG. 4. However, such a carrying case is inappropriate for the intermittent storage of a TEE transducer frequently used for examinations, at least because the case itself may become contaminated (cf. “Never store a TEE transducer in the carrying case, except to transport it”, emphasis in original, page 2-4, GUIDE). Because of the extensive cleaning and pre-reuse operations required by the TEE transducer, there is usually one or more designated areas for such operations in the hospital, clinic, or doctor's office. Typically, wall-mounted racks in the designated cleaning area end up serving as the storage area for TEE transducers between examinations.

However, these designated cleaning/disinfecting areas may be situated a considerable distance away from the location or locations where the ultrasonic transducers will be used to examine patients. For instance, in a typical mid-size hospital, the cleaning and disinfection area may be located in the basement, whereas the operating room (OR), intensive care unit (ICU), echo lab, radiology suites, or other places where endocavity ultrasound examinations are performed, may be located on the first, second, or third floor. Thus, in such a hospital, endocavity transducers will have to be transported to and from the examination area and the cleaning/disinfecting area, either with or without the rest of the ultrasound system. This naturally requires that the locations of the transducers be tracked, as well as their present disinfecting status, to ensure that each ultrasound system will have an appropriately matched disinfected transducer when an examination is about to be performed. Besides the extra work involved in such transportation and tracking, there is the risk of contamination whenever the transducers are moved from one place to another.

In addition to these problems, there may be particular storage or maintenance needs for particular transducers. For instance, the flexible shaft 230 of a TEE transducer 200 must not be bent or coiled into a circle of less than a certain diameter (typically around one foot) in order to avoid damaging the steering mechanism or shaft 230 itself. Furthermore, the distal end of the TEE transducer should be protected by a tip protector in order to prevent damage to the transducer array 342.

Therefore, there is a need for a system and apparatus for the proper maintenance and storage of endocavity transducers used by ultrasound systems. Furthermore, it should be noted that, although the problems discussed above were brought up in the context of endocavity transducers in general, and in the context of TEE transducers in particular, the problems for which the present invention is a solution may be suffered by any ultrasonic transducer which belongs in the “critical” device category (devices which contact blood, compromise tissue, or are used in a sterile field) and/or “semi-critical” device category (devices which may come into contact with mucous membranes but do not penetrate a body surface).

### SUMMARY OF THE INVENTION

One object of the present invention is to provide a receptacle appropriate for the storage, maintenance, and transportation of ultrasonic transducers used in critical or semi-critical environments.

Another object of the present invention is to provide an ultrasonic transducer receptacle which can be securably attached and detached from an ultrasound imaging system.

Yet another object of the present invention is to provide an ultrasonic transducer receptacle in which portions of the ultrasonic transducer only come into contact with a disposable liner.

Still another object of the present invention is to provide an ultrasonic transducer receptacle which has a visual means by which the present status, in terms of usage and/or cleaning/disinfecting, may be determined.

A further object of the present invention is to provide an ultrasonic transducer receptacle which is integrated into the physical structure of the ultrasound imaging system.

These and other objects are met by the present invention, a receptacle appropriate for the storage, maintenance, and transportation of ultrasonic transducers used in critical or semi-critical environments. In one aspect, the receptacle is embodied in a tray capable of being detachably secured to an ultrasound system, in which at least a portion of the ultrasonic transducer may be placed while being transported. In another aspect, the receptacle is embodied in a case which has a form-fitting cavity for holding the ultrasonic transducer, a movable or removable cover for sealing the transducer inside the case, and a handle to be used when the case is being transported.

A liner may be used to line the interior of the receptacle, thereby keeping the surfaces of the transducer separate from the interior surfaces of the receptacle. The liner may be disposable, semi-permanent, or permanent (depending on its ability to be cleaned and/or disinfected).

The movable/removable cover on the case may be transparent so that the operator can quickly determine the condition of the transducer contained within. Furthermore, a status indicator may be attached to the receptacle in order to provide the operator with information concerning the transducer.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings:

FIG. 1 is a drawing of a conventional ultrasound imaging system;

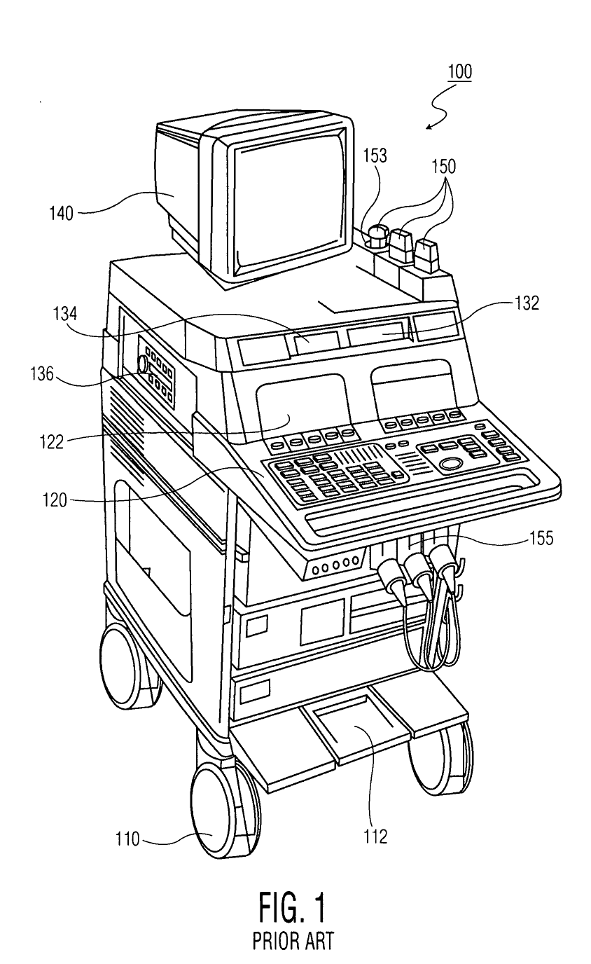

FIG. 2 is a drawing of a conventional transesophageal echo (TEE) ultrasonic transducer for use with an ultrasound system such as ultrasound system 100 in FIG. 1;

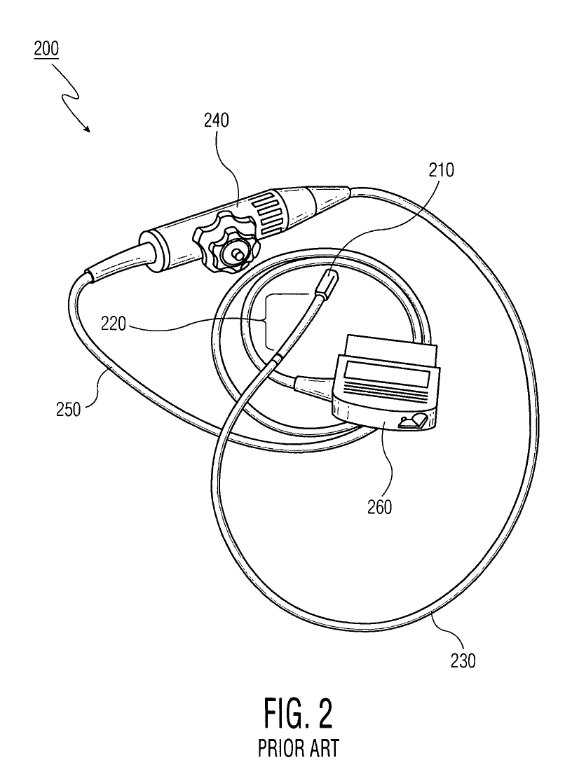

FIG. 3 is a diagram showing how the probe of a TEE transducer, such as TEE transducer 200 in FIG. 2, is inserted into the esophagus of a patient;

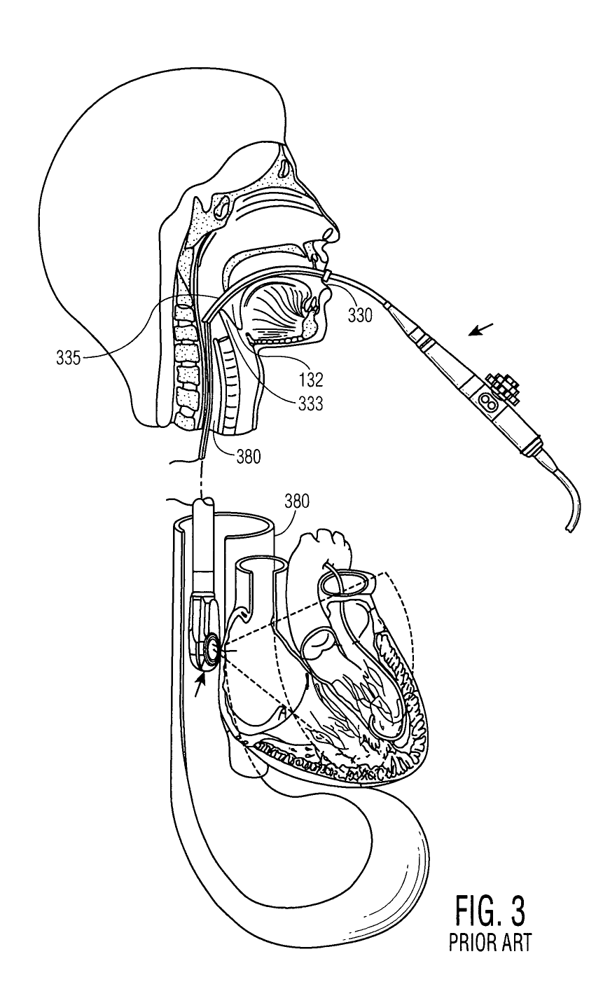

FIG. 4 is a photograph of a prior art carrying case for a TEE transducer, such as TEE transducer 200 in FIG. 2;

FIG. 5 is a drawing of an ultrasound transducer receptacle according to a first presently preferred embodiment of the present invention; and

FIG. 6 is a drawing of an ultrasound transducer receptacle according to a second presently preferred embodiment of the present invention.

### DETAILED DESCRIPTION OF THE PRESENTLY PREFERRED EMBODIMENTS

In general, the present invention is directed to a receptacle in which an ultrasonic transducer may be stored and transported, and which also provides appropriate sanitary safeguards for ultrasonic transducers used in critical or semi-critical environments. Two presently preferred embodiments are described herein: a tray which has a cavity for holding at least a portion of the ultrasonic transducer, and which is capable of being detachably secured to the ultrasound system, according to a first preferred embodiment; and a case which has a form-fitting cavity for holding the ultrasonic transducer, and which has a handle and a movable or removable cover, through or by which the operator may determine the current status of the ultrasonic transducer. These presently preferred embodiments, the tray and the case, are only two examples within the wide variety of embodiments possible in accordance with the present invention. In other words, the scope of the present invention includes embodiments which share one or more features of both the presently preferred embodiments, and embodiments which share one or more features with only one of the presently preferred embodiments.

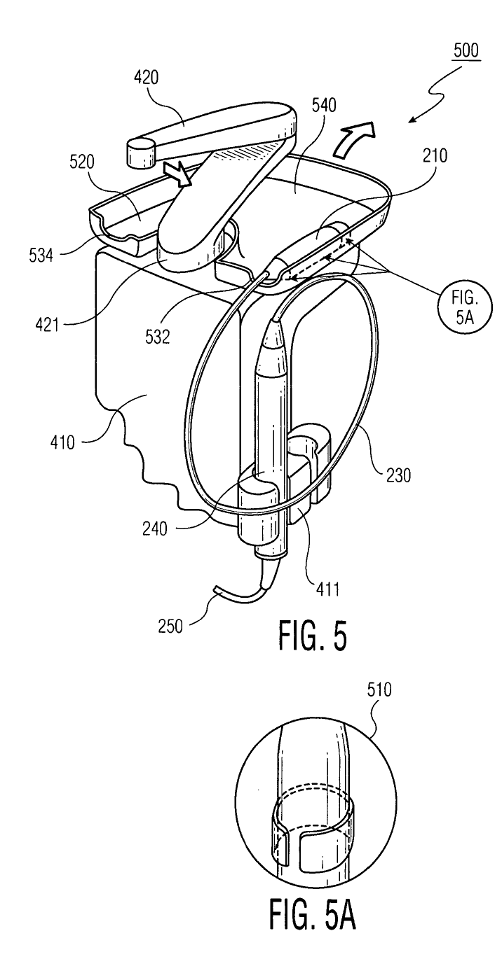

In the first presently preferred embodiment, the inventive receptacle takes the form of tray 500, as shown in FIG. 5. For convenience, only certain portions of the overall ultrasound imaging system are shown in FIG. 5. The portions of the ultrasound system shown in FIG. 5 include a pedestal 410 and an articulating monitor arm 420 which holds the system's monitor (not shown in FIG. 5) and enables the operator to move the monitor into an appropriate position. Portions of transducer probe 200 can also be seen in FIG. 5, including probe handle 240, which is set into probe handle holder 411 on pedestal 410, and probe 210, which is resting in a portion of tray 500.

As can be seen in FIG. 5, tray 500 is constructed to complement the architecture of the area to which it is attached. For example, the profile of tray 500 in the horizontal plane is roughly in the shape of an upside-down “U” in order that tray 500 may fit atop pedestal 410 yet still fit around the base 421 of monitor arm 420. In this manner, the shape of tray 500 complements the overall structure of the ultrasound system, and does not protrude from either side of the ultrasound system, nor above the monitor. Because of its complementary shape, tray 500 will not obstruct a technician's view when moving the entire ultrasound system, nor is tray 500 likely to catch on a doorway or other object while the entire ultrasound system is being moved. It should be noted that tray 500 is only one example of complementarity according to the present inventive receptacle. In other words, the manner in which another receptacle may complement another ultrasound system may be completely different, depending on the specific shape of the other ultrasound system. Furthermore, a receptacle according to the present invention does not have to be resting on a planar surface of the ultrasound system (as tray 500 is resting on pedestal 410), but may rather be contained within a portion of the ultrasound system, for example, or be attached to a side of the ultrasound system.

Tray 500 is constructed of a material which is capable of being cleaned, disinfected, and/or sterilized (thereby making tray 500 reusable). However, in other embodiments of the present invention, it is possible for the receptacle to be constructed from a material which is disposable, i.e., an embodiment where the receptacle itself is disposable or semi-permanent. Tray 500 preferably weighs less than a maximum of about 10 pounds when empty.

Tray 500 is removably attached to pedestal 410. In FIG. 5, the means of removable attachment comprise portions on the bottom of tray 500 which engage portions on the top of pedestal 410 when tray 500 is moved and locked into place. The removable attachment means may comprise one or more protrusions formed on the top surface of pedestal which fit into one or more matching receiving means, such as tracks, on the bottom surface of tray 500. In other embodiments, the removable attachment means may comprise any form of mechanical means, including, but not limited to, one or more snaps, one or more latches, a notch and detent system, or a system of mounting brackets. In any of these embodiments, the attachment means is intended to keep tray 500 secure enough so that tray 500 will not be dislodged by the typical movements of the entire ultrasound system when it is being moved from place to place.

In FIG. 5, the probe 210 at the distal end of transducer 200 is sitting in a portion of tray 500. Probe 210 is held in place by clip 510 in order to prevent probe 210 from falling out of tray 500. As shown in FIG. 5, clip 510 may be used to hold any portion of probe 210, as long as the sensitive portions of probe 210, such as transducer array 342, are adequately protected. A protective tip may cover probe 210 while probe 210 is clipped into tray 500. Probe 210 may be covered and clipped to tray 500 immediately after being disinfected.

The tray embodiment shown in FIG. 5 can be used for carrying portions or the entirety of transducer 200 to and from a cleaning/disinfecting area. For example, after an examination, probe 210 and flexible shaft 230 may be disconnected from probe handle 240 and placed in tray 500, which is then detached from pedestal 410 and brought to the cleaning/disinfecting area. It may be desireable to separate these portions from the remaining portions of the transducer 200 because probe 210 and flexible shaft 230 need a higher level of disinfection than probe handle 240 or connector cable 250 because probe 210 and flexible shaft 230 were inserted into the patient's body. Typically, probe 210 and flexible shaft 230 have to be immersed in a glutaraldehyde solution, whereas the probe handle 240 and connector cable 250 need only be cleaned with soap and water (and may, in fact, be damaged if immersed in the glutaraldehyde solution).

Preferably, the entire transducer 200 is placed in tray 500 for transport to the cleaning/disinfecting area, where all the component parts of transducer 200 are appropriately cleaned and disinfected. When tray 500 is holding the entire transducer, probe handle 240 is held in portion 520 across from where probe 210 is clipped, and flexible shaft 230 juts out from tray 500. Specifically, shaft 340 runs out from probe 210 at trap lip 532 and re-enters tray 500 so that the portion entering probe handle 240 rests on at tray lip 534. Of course, other embodiments may wish to avoid having any portion of the transducer hanging out of tray 500. The remaining portions of transducer 200, connector cable 250 and connector plug 260 (not shown in FIG. 5), are placed in section 540 of tray 500. After being cleaned, disinfected, and appropriately dried, transducer 200 may be stored in tray 500, and await its next use. Tray 500 may also be used to carry other ultrasound system accessories, such as the mouth guard and ECG pads.

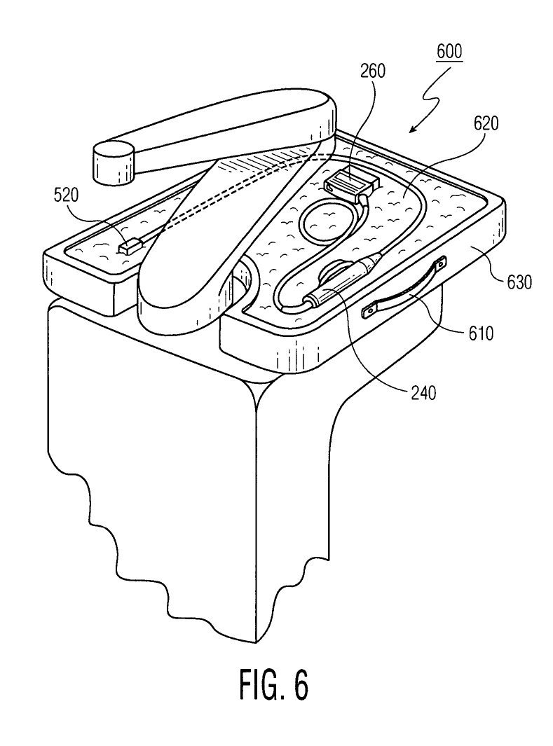

In order to clarify the cleaning/disinfecting status of the transducer 200 lying in tray 500, a status indicator may be used to indicate whether it is currently ready to be used, whether it has just been used, or whether it has been in storage, and for how long (as it may require another cycle of cleaning/disinfecting before use). Systems of status identification according to the present invention will be described in greater detail in reference to the second preferred embodiment, described below with reference to FIG. 6.

In the first preferred embodiment of the present invention, as shown in FIG. 5, a disposable liner may be used to line the inside of tray 500 before transducer 200 is placed back into tray 500 after a cleaning/disinfecting. Preferably, this is done after each cleaning/disinfecting procedure. After each examination, the disposable liner is left in place until tray 500 arrives at the cleaning/disinfecting area, where it is removed and either cleaned/disinfected or thrown out. In other embodiments, the liner may be permanent or semi-permanent, with the intention that the liner be cleaned/disinfected after each examination. Furthermore, a removable cover may be locked onto the top of tray 500 after each cleaning/disinfecting in order to ensure that the transducer remains relatively clean while being stored. Movable and removable covers according to the present invention will be described in greater detail in reference to the second preferred embodiment, described below with reference to FIG. 6. In the case of tray 500, such a cover might comprise a clear plastic material which is spread over the top of tray 500 in order to seal in transducer 200. The plastic seal remains intact until the next time the transducer is used, when it is removed and thrown out.

In the second presently preferred embodiment, the inventive receptacle takes the form of a case 600, as shown in FIG. 6. The case in FIG. 6 has roughly the same profile as the tray in FIG. 5, but also includes a cover (not shown in FIG. 6), and a handle 610 which can be used to hold case 600 when transporting the transducer separately from the rest of the ultrasound imaging system. The cover is either removable or movable (i.e., capable of both sealing in the contents of case 600 and moving out of the way so that the operator may access the contents). Examples of movable covers include, but are not limited to, covers connected to case 600 by hinges, covers which lock onto, snap onto, slide onto, or form another type of friction seal with case 600, and covers which are sealed onto case 600 by a vacuum seal.

Preferably, the cover of case 600 is substantially transparent so that the operator may easily observe the transducer contained therein in order to determine general status and/or level of contamination of the transducer. In addition, a status indicator may be incorporated into the cover or into another part of case 600. The status indicator would provide information to anyone viewing the outside of case 600 as to the transducer stored within. Such information may include, for example, the state of disinfection and/or cleaning, the appropriate routing for the transducer, the owner of the transducer, the safety test status of the transducer, the repair status of the transducer, or any other information of importance concerning the transducer.

The status indicator may be implemented, for example, by lettering, symbols, and/or color codes on a designated rewritable surface located on the transparent cover. As another example, a system of status indication could be used in which removable labels (with lettering, symbols, and/or color codes) are placed on, and removed from, the cover after specified acts, events, or periods of time. The status indicator could also be implemented by mechanical means (such as movable tabs in slots) or electronic means (such as an LED or LCD display with appropriate controls) attached to the cover of case 600. Other embodiments of the present invention may locate the status indicator on a different surface of case 600, or may implement the status indicator by another means.

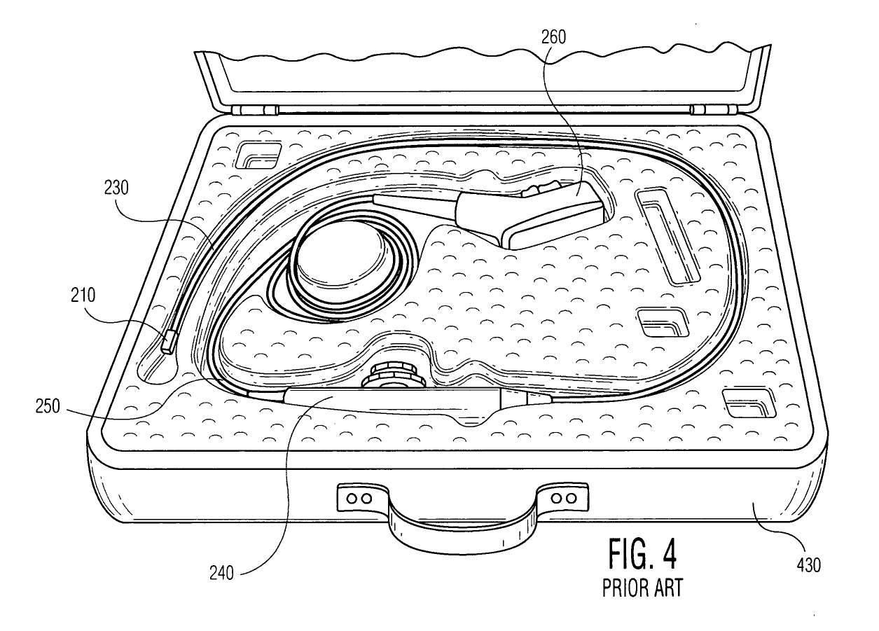

The interior 620 of case 600 has a form-fitting cavity for the transducer. The form-fitting cavity would be similar in shape to the cavity shown in the prior art case of FIG. 4 and may be constructed of, for example, foam or plastic. Similar to tray 500, the form-fitting cavity of case 600 may have a liner for keeping the form-fitting cavity and/or the transducer clean and disinfected, and the liner may be disposable, permanent, or semi-permanent. The outside surface 630 of case 600 may be constructed of either hard material or soft material, which may depend on the material used for the form-fitting cavity in the interior 620 of case 600.

While there have shown and described and pointed out fundamental novel features of the invention as applied to two preferred embodiments thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

1. A receptacle for storing an ultrasonic transducer for an ultrasound imaging system comprising:
a receiving section for receiving at least a portion of the ultrasonic transducer; and
an attachment system suitable for an operator to easily attach the receptacle to the ultrasound system and easily detach the receptacle from the ultrasound system;
wherein the receptacle is suitable for transporting the at least a portion of the ultrasonic transducer to and/or from a cleaning and/or disinfecting area; and
wherein the shape of the receptacle is such that it complements the structural design of the ultrasound imaging system.

2. The receptacle of claim 1, wherein the receiving section defines a form-fitting cavity for receiving the ultrasonic transducer.

3. The receptacle of claim 1, wherein a liner is applied to the receiving section of the receptacle.

4. The receptacle of claim 3, wherein the liner applied to the receiving section of the receptacle is disposable.

5. The receptacle of claim 1, wherein the attachment system comprises at least one of at least one snap, at least one latch, a notch and detent system, a friction seal system, or a vacuum seal system.

6. The receptacle of claim 1, wherein the shape of the receptacle complements the structural design of the ultrasound imaging system in that no portion of the receptacle protrudes substantially further out from the ultrasound system than any portion of the ultrasound system.

7. The receptacle of claim 1, wherein the shape of the receptacle complements the structural design of the ultrasound imaging system in that the receptacle fits at least one of on a planar surface of the ultrasound system, on a side of the ultrasound system, or in a recess of the ultrasound system.

8. The receptacle of claim 1, wherein the shape of the receptacle complements the structural design of the ultrasound imaging system in that the receptacle does not obstruct an operator when either performing an imaging operation on the ultrasound system or moving the ultrasound system from one location to another.

9. The receptacle of claim 1, further comprising:
a status indicator.

10. The receptacle of claim 9, wherein the status indicator is located on an exterior of the receptacle.

11. The receptacle of claim 9, wherein the status indicator indicates information comprising at least one of: a state of disinfection and/or cleaning, an appropriate routing for the transducer, an owner of the transducer, a safety test status of the transducer, or a repair status of the transducer.

12. The receptacle of claim 1, further comprising:
a cover for at least partially covering the at least a portion of the ultrasonic transducer.

13. The receptacle of claim 12, wherein the cover is comprised of substantially transparent material.

14. The receptacle of claim 12, wherein the cover is at least one of removable from the receptacle or movable relative to the receptacle such that an operator may access the contents of the receptacle.

15. The receptacle of claim 1, further comprising:
a handle suitable for a person to carry the receptacle.

16. The receptacle of claim 1, wherein the ultrasonic transducer is used in at least one of a semi-critical environment and critical environment.

17. The receptacle of claim 16, wherein the ultrasonic transducer is one of an endocavity transducer and an intraoperative transducer.

18. The receptacle of claim 1, wherein the receptacle comprises at least one of a tray and a case.

19. An ultrasound imaging system comprising:
an ultrasonic transducer for use in at least one of a critical environment and a semi-critical environment;
a receptacle suitable for storing and transporting at least a portion of the ultrasonic transducer, wherein said receptacle comprises:
a receiving section for receiving the at least a portion of the ultrasonic transducer;
an attachment system suitable for an operator to easily attach the receptacle to the ultrasound system and easily detach the receptacle from the ultrasound system;
a cover for at least partially covering the at least a portion of the ultrasonic transducer;
a handle suitable for a person to carry the receptacle; and
a status indicator;
wherein a liner may be applied to the receiving section.

20. The ultrasound imaging system of claim 19, wherein the status indicator indicates information comprising at least one of: a state of disinfection and/or cleaning, an appropriate routing for the transducer, an owner of the transducer, a safety test status of the transducer, or a repair status of the transducer.
